(19) United States
(12) Patent Application Publication
(10) Pub. No.: US 2023/0301991 A1
(43) Pub. Date: Sep. 28, 2023

(54) COMPOUNDS FOR THE TREATMENT OF VIRAL INFECTIONS

(71) Applicant: MERCK PATENT GMBH, DARMSTADT (DE)

(72) Inventors: Ulrich BETZ, Darmstadt (DE); Thomas FUCHSS, Darmstadt (DE)

(73) Assignee: MERCK PATENT GMBH, DARMSTADT (DE)

(21) Appl. No.: 18/033,724

(22) PCT Filed: Oct. 22, 2021
(86) PCT No.: PCT/EP2021/079300

§ 371 Date: Apr. 25, 2023

(30) Foreign Application Priority Data
Oct. 26, 2020 (EP) 20203798.2
Jan. 29, 2021 (EP) 21154343.4

(51) Int. Cl. A61K 31/497; A61K 45/06; A61P 31/14

(52) U.S. Cl. CPC A61K 31/497; A61K 45/06; A61P 31/14

(57) ABSTRACT

The present invention encompasses ATR inhibitor for use in the treatment of coronavirus infections, including COVID-19, alone or in combination with one or more additional therapeutic agents.

### TECHNICAL FIELD OF THE INVENTION

The present invention provides for the use of ataxia telangiectasia and Rad3-related protein (ATR) inhibitors in the treatment of coronavirus infections, including SARS-CoV infections such as COVID-19.

### BACKGROUND OF THE INVENTION

ATR kinase is a protein kinase involved in cellular responses to certain forms of DNA damage (e.g., double strand breaks and replication stress). ATR kinase acts with ATM (“ataxia telangiectasia mutated”) kinase and many other proteins to regulate a cell's response to double strand DNA breaks and replication stress, commonly referred to as the DNA Damage Response (“DDR”). The DDR stimulates DNA repair, promotes survival and stalls cell cycle progression by activating cell cycle checkpoints, which provide time for repair. Without the DDR, cells are much more sensitive to DNA damage and readily die from DNA lesions induced by endogenous cellular processes such as DNA replication or exogenous DNA damaging agents commonly used in cancer therapy.

ATR is upregulated in a variety of cancer cell types and plays a key role in DNA repair, cell cycle progression, and survival it is activated by DNA damage caused during DNA replication-associated stress. Inhibitors of ataxia telangiectasia and rad3-related (ATR) kinase prevents ATR-mediated signaling in the ATR-checkpoint kinase 1 (Chk1) signaling pathway. This prevents DNA damage checkpoint activation, disrupts DNA damage repair, and induces tumor cell apoptosis. ATR inhibitors are in clinical development of various solid tumors, e.g. small-cell cancers, urothelial carcinoma and ovarian cancer.

### Coronaviruses

Coronaviruses (CoVs) are positive-sense, single-stranded RNA (ssRNA) viruses of the order Nidovirales, in the family Coronaviridae. There are four sub-types of coronaviruses—alpha, beta, gamma and delta—with the Alphacoronaviruses and Betacoronaviruses infecting mostly mammals, including humans. Over the last two decades, three significant novel coronaviruses have emerged which jumped from a non-human mammal hosts to infect humans: the severe acute respiratory syndrome (SARS-CoV-1) which appeared in 2002, Middle East respiratory syndrome (MERS-CoV) which appeared in 2012, and COVID-19 (SARS-CoV-2) which appeared in late 2019. By mid-Oct. of 2020, over 40 million people are known to have been infected, and over 1 million people have died. Both numbers likely represent a significant undercount of the devastation wrought by the disease.

### COVID-19

SARS-CoV-2 closely resembles SARS-CoV-1, the causative agent of SARS epidemic of 2002-03 (Fung, et al, Annu. Rev. Microbiol. 2019. 73:529-57). Severe disease has been reported in approximately 15% of patients infected with SARS-CoV-2, of which one third progress to critical disease (e.g. respiratory failure, shock, or multiorgan dysfunction (Siddiqi, et al, J. Heart and Lung Trans. (2020), doi: https://doi.org/10.1016/j.healun.2020.03.012, Zhou, et al, Lancet 2020; 395: 1054-62. https://doi.org/10.1016/S0140-6736(20)30566-3). Fully understanding the mechanism of viral pathogenesis and immune responses triggered by SARS-CoV-2 would be extremely important in rational design of therapeutic interventions beyond antiviral treatments and supportive care. Much is still being discovered about the various ways that COVID-19 impacts the health of the people that develop it.

Severe acute respiratory syndrome (SARS)-Corona Virus-2 (CoV-2), the etiologic agent for coronavirus disease 2019 (COVID-19), has caused a pandemic affecting almost eight million people worldwide with a case fatality rate of 2-4% as of June 2020. The virus has a high transmission rate, likely linked to high early viral loads and lack of pre-existing immunity (He, et. al, Nat Med 2020 https://doi.org/10.1038/s41591-020-0869-5). It causes severe disease especially in the elderly and in individuals with comorbidities. The global burden of COVID-19 is immense, and therapeutic approaches are increasingly necessary to tackle the disease. Intuitive anti-viral approaches including those developed for enveloped RNA viruses like HIV-1 (lopinavir plus ritonavir) and Ebola virus (remdesivir) have been implemented in testing as investigational drugs (Grein et al, NEJM 2020 https://doi.org/10.1056/NEJMoa2007016: Cao,et al, NEJM 2020 DOI: 10.1056/NEJMoa2001282). But given that many patients with severe disease present with immunopathology, host-directed immunomodulatory approaches are also being considered, either in a staged approach or concomitantly with antivirals (Metha, et al, The Lancet 2020; 395(10229) DOI: https://doi.org/10.1016/S0140-6736(20)30628-0, Stebbing, et al, Lancet Infect Dis 2020. https://doi.org/10.1016/S1473-3099(20)30132-8).

While there are many therapies being considered for use in treatment of COVID-19, there are as yet no approved medications to treat the disease, and no vaccine available. To date, treatment typically consists only of the available clinical mainstays of symptomatic management, oxygen therapy, with mechanical ventilation for patients with respiratory failure. Thus, there is an urgent need for novel therapies to address the different stages of the SARS-CoV-2 infectious cycle (Siddiqi, et al.).

### Human Cytomegalovirus (HCMV)

Human cytomegalovirus (HCMV) (also human betaherpesvirus 5 (HHV-5), cytomegalovirus (ZMV), cytomegalovirus (CMV)) is an enveloped, double-stranded DNA virus (dsDNA), belongs to the family Herpesviridae, genus Cytomegalovirus and is distributed worldwide. Transmission occurs via saliva, urine, sperm secretions, and during blood transfusion.

Human cytomegalovirus (HCMV) is a major cause of birth defects and opportunistic infections in immunosuppressed individuals, and a possible cofactor in certain cancers, organ transplant patients under immunosuppressive therapy are at high risk for viral infections; activation of a latent virus as well as donor or community acquired primary infections can cause significant complications including graft rejection, morbidity, and mortality Herpesviruses (e.g HCMV, HSV1), polyomaviruses (e g. BKV and JCV), hepatitis viruses (HBV and HCV) and respiratory viruses (c.g. influenza A, adenovirus) are the 4 major viral classes infecting these patients. Cytomegalovirus (HCMV) is the most prevalent post-transplant pathogen, HCMV can infect most organs, and despite the availability of HCMV antivirals such as acyclovir or ganciclovir, nephrotoxic side effects and increasing rates of drug-resistance significantly reduce graft and patient survival In addition, HCMV-mediated immune modulation can reactivate distinct latent viruses carried by most adults.

### BRIEF DESCRIPTION OF THE DRAWINGS

FIG. 1 shows a graph depicting the influence of Compound 1 on the viral replication in Calu-3 cells infected with MERS (red dots) and on cell viability (black dots).

FIG. 2 shows a graph depicting the influence of Compound 1 on the viral replication in Calu-3 cells infected with SARS-CoV-1 (red dots) and on cell viability (black dots).

FIG. 3 shows a graph depicting the influence of Compound 1 on the viral replication in Calu-3 cells infected with SARS-CoV-2 (red dots) and on cell viability (black dots).

FIG. 4 shows a graph depicting the influence of remdesivir on the viral replication in Calu-3 cells infected with SARS-CoV-2 (red dots) and on cell viability (black dots).

FIG. 5 shows a graph depicting the influence of Compound 1 on the viral replication in human foreskin fibroblasts infected with Cytomegalovirus (black dots) and on cell viability (gray squares).

FIG. 6 shows a graph depicting the influence of acyclovir on the viral replication in human foreskin fibroblasts infected with Cytomegalovirus (black dots) and on cell viability (gray squares).

FIG. 7 shows a graph depicting the influence of Compound 1 on the viral replication in human foreskin fibroblasts infected with Cytomegalovirus (black dots) and on cell viability (gray squares).

### SUMMARY OF THE INVENTION

In a first embodiment, the invention provides the ATR inhibitors of the invention for use in the treatment of viral infections in a subject in need thereof. In one aspect of this embodiment, the viral infection is a single-strand RNA viral infection. In another aspect of this embodiment, the viral infection is a coronavirus infection. In a further aspect of this embodiment, the viral infection is a SARS-CoV1, MERS-CoV, or SARS-CoV-2 infection. In a final aspect of this embodiment, the viral infection is a SARS-CoV-2 infection.

A second embodiment is a method of treating a coronavirus infection in a subject in need thereof, comprising administering an effective amount of an ATR inhibitor, or a pharmaceutically acceptable salt thereof, to the subject. In one aspect of this embodiment, the administration of compound

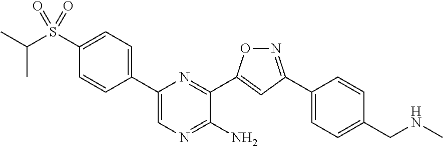 [SMILES: CNCc1ccc(-c2cc(-c3nc(-c4ccc(S(=O)(=O)C(C)C)cc4)cnc3N)on2)cc1]

reduces the viral load in the subject. In one aspect of this embodiment, the ATR inhibitor is administered prior to COVID-19 pneumonia development. In a further aspect of this embodiment, the subject has a mild to moderate SARS-CoV-2 infection. In an additional aspect of this embodiment, the subject is asymptomatic at the start of the administration regimen.

In a further embodiment, the viral infection is a double-strand DNA viral infection. In another aspect of this embodiment, the viral infection is a HCMV infection. A preferred embodiment is a method of treating a Cytomegalovirus infection in a subject in need thereof, comprising administering an effective amount of an ATR inhibitor, or a pharmaceutically acceptable salt thereof, to the subject. In one aspect of this embodiment, the administration of compound

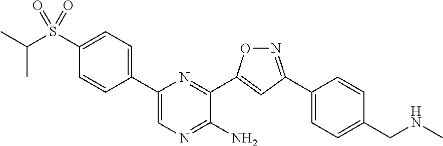 [SMILES: CNCc1ccc(-c2cc(-c3nc(-c4ccc(S(=O)(=O)C(C)C)cc4)cnc3N)on2)cc1]

reduces the viral load in the subject.

The invention of this patent application can also be summarized as follows: An ATR inhibitor or a pharmaceutically acceptable salt thereof for use in the treatment of a coronavirus infection. Use of an ATR inhibitor or a pharmaceutically acceptable salt thereof in the manufacture of a medicament for the treatment of a coronavirus infection. An ATR inhibitor or a pharmaceutically acceptable salt thereof for use in the treatment of a Cytomegalovirus infection. Use of an ATR inhibitor or a pharmaceutically acceptable salt thereof in the manufacture of a medicament for the treatment of a Cytomegalovirus infection.

### DETAILED DESCRIPTION

Coronaviruses comprise a diverse group of enveloped positive-strand RNA viruses that are responsible for several human diseases, most notably the severe acute respiratory syndrome (SARS) epidemics in 2003 and 2020.

Infectious Bronchitis Virus (IBV), a highly infectious avian gamma-coronavirus, that primarily targets cells of the respiratory tract, can inhibit cell growth by inducing cell cycle arrest in G2 and S-phases in infected cells (Dove B. et al.: Cell cycle perturbations induced by infection with the coronavirus infectious bronchitis virus and their effect on virus replication. J. Virol. 80, 4147-4156, 2006; Li, F. Q. et al.: Cell cycle arrest and apoptosis induced by the coronavirus infectious bronchitis virus in the absence of p53. Virology 365, 435-445, 2007). Xu et al. have shown that activation of the cellular DNA damage response is one of the mechanisms exploited by Coronavirus to induce cell cycle arrest and that suppression of the ATR kinase activity by chemical inhibitors and siRNA-mediated knockdown of ATR reduced the IBV-induced ATR signaling and inhibited the replication of IBV (Xu L. H. et al.: Coronavirus Infection Induces DNA Replication Stress Partly through Interaction of Its Nonstructural Protein 13 with the p125 Subunit of DNA Polymerase J Biol Chem 286: 39546-39559, 2011).

Recent papers have suggested a correlation between SARS-CoV-2 viral load, symptom severity and viral shedding (He, et al; Liu, et al, Lancet Infect Dis 2020. https://doi.org/10.1016/S1473-3099(20)30232-2). Some antiviral drugs administered at symptom onset to blunt coronavirus replication are in the testing phase (Grein, et al; Taccone, et al), but as yet none have shown much promise. Being able to slow the viral reproduction in the early stages of infection may allow the subject to avoid severe disease.

It is believed that compounds of the invention inhibit the coronavirus induced DNA damage response and the replication of the coronavirus in the host by inhibiting the virus induced activation of cellular DNA damage response. It is conceived that compounds of the invention may inhibit nucleic acid replication, virus assembly, new virus particle transport, and/or virus release. The result of administration of a compound of the invention is to reduce viral replication, which in turn will reduce viral load, and reduce the severity of disease.

Whatever the exact mechanism of action for the antiviral properties of the compounds of the invention, it is proposed that administration thereof may have one or more clinical benefits, as described further herein.

“COVID-19” is the name of the disease which is caused by a SARS-CoV-2 infection. While care was taken to describe both the infection and disease with accurate terminology, “COVID-19” and “SARS-CoV-2 infection” are meant to be roughly equivalent terms.

As of the writing of this application, the determination and characteristics of the severity of COVID-19 patients/symptoms has not been definitively established. However, in the context of this invention, “mild to moderate” COVID-19 occurs when the subject presents as asymptomatic or with less severe clinical symptoms (e.g., low grade or no fever (<39.1° C.), cough, mild to moderate discomfort) with no evidence of pneumonia, and generally does not require medical attention. When “moderate to severe” infection is referred to, generally patients present with more severe clinical symptoms (e.g., fever >39.1° C., shortness of breath, persistent cough, pneumonia, etc.). As used herein “moderate to severe” infection typically requires medical intervention, including hospitalization. During the progression of disease, a subject can transition from “mild to moderate” to “moderate to severe” and back again in one course of bout of infection.

Treatment of COVID-19 using the methods of this invention include administration of an effective amount of an ATR inhibitor of the invention at any stage of the infection to prevent or reduce the symptoms associated therewith. Typically, subjects will be administered an effective amount of an ATR inhibitor of the invention after definitive diagnosis and presentation with symptoms consistent with a SARS-CoV2 infection, and administration will reduce the severity of the infection and/or prevent progression of the infection to a more severe state. The clinical benefits upon such administration is described in more detail in the sections below.

### 1. Compounds and Definitions

One embodiment is use of a compound

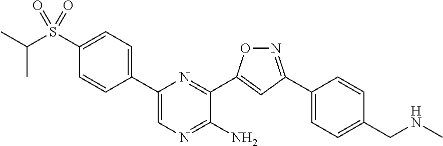 [SMILES: CNCc1ccc(-c2cc(-c3nc(-c4ccc(S(=O)(=O)C(C)C)cc4)cnc3N)on2)cc1]

3-[3-(4-Methylaminomethyl-phenyl)-isoxazol-5-yl]-5-[4-(propane-2-sulfonyl)-phenyl]-pyrazin-2-ylamine (hereinafter also referred to as “Compound 1”), or a pharmaceutically acceptable salt thereof for the treatment of a viral infection.

Compound 1 is disclosed in WO 2010/071837 A1 as Compound IIA-7 (Example 57A).

The above compounds may either be used in their free forms or as pharmaceutically acceptable salts. The free compounds may be converted into the associated acid-addition salt by reaction with an acid, for example by reaction of equivalent amounts of the base and the acid in an inert solvent, such as, for example, ethanol, and subsequent evaporation. Suitable acids for this reaction are, in particular, those which give physiologically acceptable salts, such as, for example, hydrogen halides (for example hydrogen chloride, hydrogen bromide or hydrogen iodide), other mineral acids and corresponding salts thereof (for example sulfate, nitrate or phosphate and the like), alkyl- and monoarylsulfonates (for example ethanedisulfonate (edisylate), toluenesulfonate, napthalene-2-sulfonate (napsylate), benzenesulfonate) and other organic acids and corresponding salts thereof (for example fumarate, oxalate, acetate, trifluoroacetate, tartrate, maleate, succinate, citrate, benzoate, salicylate, ascorbate and the like.

Exemplary embodiments of the pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

In one embodiment Compound 1 is used in the form of one of its crystalline forms as described in Examples 10 to 14 of WO 2013/049726 A2. Preferably the crystalline form is the free base of Compound 1 as described in Example 10 or its hydrochloride salt as described in Example 11. Such forms are:

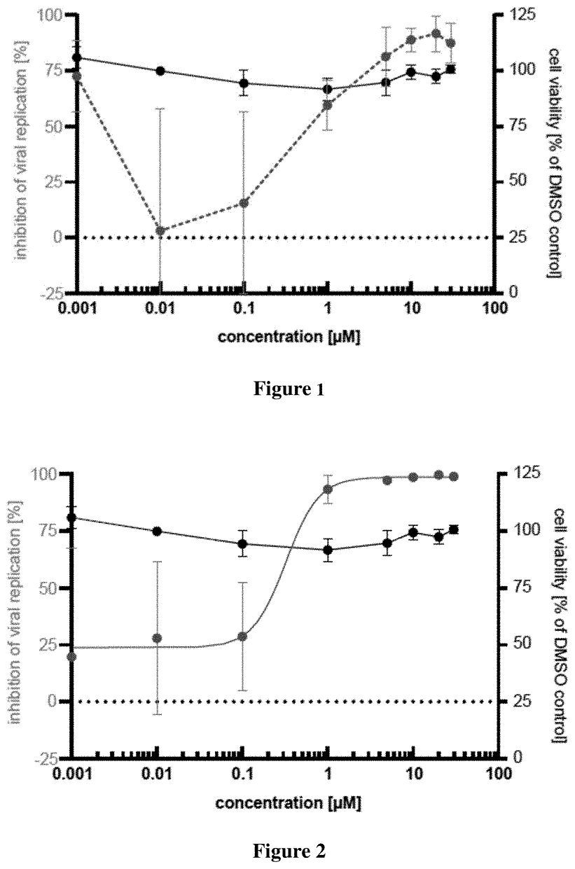

A solid form of Compound 1, wherein the form is crystalline Compound 1 free base having a monoclinic crystal system and a P2₁/n space group, such solid form having the following unit cell dimensions in Å when measured at 120 K: a=8.9677 (1) Å, b=10.1871 (1) Å, c=24.5914 (3) Å, such solid form, characterized by a weight loss of about 1.9% in a temperature range of from about 25° C. to about 215° C. as determined by thermogravimetric (TGA) analysis, such solid form, characterized by one or more peaks expressed in 2-theta ±0.2 at about 14.2, 25.6, 18.1, 22.0, and 11.1 degrees in an X-ray powder diffraction pattern obtained using Cu K alpha radiation, such solid form, characterized as having an X-ray powder diffraction pattern substantially the same as that shown in FIG. 1a of WO 2013/049726 A2;

A solid form of Compound 1, wherein the form is crystalline Compound 1⋅ HC1, such solid form having a monoclinic crystal system and a P2₁/n space group, such solid form, characterized by a weight loss of about 1.1% in a temperature range of from about 25° C. to about 100° C., and by a further weight loss of about 0.8% in a temperature range of from about 110° C. to about 240° C. as determined by thermogravimetric (TGA) analysis, such solid form, characterized by one or more peaks expressed in 2-theta ±0.2 at about 13.5, 28.8, 15.0, 18.8, and 15.4 degrees in an X-ray powder diffraction pattern obtained using Cu K alpha radiation, such solid form, characterized as having an X-ray powder diffraction pattern substantially the same as that shown in FIG. 1b of WO 2013/049726 A2.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. In some embodiments, the group comprises one or more deuterium atoms.

### 2. Uses, Formulation and Administration

The term “patient” or “subject”, as used herein, means an animal, preferably a human. However, “subject” can include companion animals such as dogs and cats. In one embodiment, the subject is an adult human patient. In another embodiment, the subject is a pediatric patient. Pediatric patients include any human which is under the age of 18 at the start of treatment. Adult patients include any human which is age 18 and above at the start of treatment. In one embodiment, the subject is a member of a high-risk group, such as being over 65 years of age, immunocompromised humans of any age, humans with chronic lung conditions (such as, asthma, COPD, cystic fibrosis, etc.), and humans with other co-morbidities. In one aspect of this embodiment, the other co-morbidity is obesity, diabetes, and/or hypertension.

Compositions of the present invention are administered orally, parenterally, by inhalation spray, topically, rectally, nasally, buccally, vaginally or via an implanted reservoir. Preferably, the compositions are administered orally. In one embodiment, the oral formulation of a compound of the invention is a tablet or capsule form. In another embodiment, the oral formulation is a solution or suspension which may be given to a subject in need thereof via mouth or nasogastric tube. Any oral formulations of the invention may be administered with or without food. In some embodiments, pharmaceutically acceptable compositions of this invention are administered without food. In other embodiments, pharmaceutically acceptable compositions of this invention are administered with food.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

In one embodiment, the total amount of ATR inhibitor administered to the subject in need thereof is between about 20 mg to about 2000 mg, which can be applied once to four times per day to once every week. In one aspect of this embodiment, the total amount of ATR inhibitor administered is between about 50 mg and about 350 mg per day and is preferably administered once a day.

In another embodiment, the ATR inhibitor is administered once a day. In another aspect of this embodiment, the ATR inhibitor is administered twice a day.

In any of the above embodiments, the ATR inhibitor is administered for a period of about 7 day to about 28 days. In one aspect of any of the above embodiments, the ATR inhibitor is administered for about 14 days.

In one embodiment of the invention, the subject is suffering from COVID-19 pneumonia. In one embodiment of this invention, the subject is suffering from one or more symptoms selected from chest congestion, cough, blood oxygen saturation (SpO₂) levels below 94%, shortness of breath, difficulty breathing, fever, chills, repeated shaking with chills, muscle pain and/or weakness, headache, sore throat and/or new loss of taste or smell.

In one embodiment of the invention, the subject is being treated inpatient in a hospital setting. In another embodiment, the subject is being treated in an outpatient setting. In one aspect of the preceding embodiments, the subject may continue administration of the ATR inhibitors after being transitioned from being treated from an inpatient hospital setting to an outpatient setting.

In one embodiment, the administration of the ATR inhibitors results in one or more clinical benefit. In one aspect of this embodiment, the one or more clinical benefit is selected from the group comprising: reduction of duration of a hospital stay, reduction of the duration of time in the Intensive Care Unit (ICU), reduction in the likelihood of the subject being admitted to an ICU, reduction in the rate of mortality, reduction in the likelihood of kidney failure requiring dialysis, reduction in the likelihood of being put on non-invasive or invasive mechanical ventilation, reduction of the time to recovery, reduction in the likelihood supplemental oxygen will be needed, improvement or normalization in the peripheral capillary oxygen saturation (SpO₂ levels) without mechanical intervention, reduction of severity of the pneumonia as determined by chest imaging (eg, CT or chest X ray), reduction in the cytokine production, reduction of the severity of acute respiratory distress syndrome (ARDS), reduction in the likelihood of developing ARDS, clinical resolution of the COVID-19 pneumonia and improvement of the PaO₂/FiO₂ ratio in the subject.

In another embodiment, the one or more clinical benefits includes the improvement or normalization in the peripheral capillary oxygen saturation (SpO₂ levels) in the subject without mechanical ventilation or extracorporeal membrane oxygenation.

In a further embodiment, the one of more clinical benefits is reduction in the likelihood of being hospitalized, reduction in the likelihood of ICU admission, reduction in the likelihood being intubated (invasive mechanical ventilation), reduction in the likelihood supplemental oxygen will be needed, reduction in the length of hospital stay, reduction in the likelihood of mortality, and/or a reduction in likelihood of relapse, including the likelihood of rehospitalization.

The invention also provides a method of treating a viral infection in a subject in need thereof comprising administering an effective amount of a compound of the invention to the subject. An amount effective to treat or inhibit a viral infection is an amount that will cause a reduction in one or more of the manifestations of viral infection, such as viral lesions, viral load, rate of virus production, and mortality as compared to untreated control subjects.

One embodiment of the invention is a method of treating a coronavirus infection in a subject in need thereof, comprising administering an effective amount of an ATR inhibitor, or a pharmaceutically acceptable salt thereof, to the subject. In one aspect of this embodiment, the subject is infected with SARS-CoV-2. In another aspect of this embodiment, the administration of the ATR inhibitor results in the reduction of the viral load in the subject.

In one embodiment, the ATR inhibitor is administered prior to COVID-19 pneumonia developing. In another embodiment, the subject has a mild to moderate SARS-CoV-2 infection. In a further embodiment, the subject is asymptomatic at the start of the administration regimen. In another embodiment, the subject has had known contact with a patient who has been diagnosed with a SARS-CoV-2 infection. In an additional embodiment, the subject begins administration of the ATR inhibitor prior to being formally diagnosed with COVID-19.

One embodiment is a method of treating a subject with COVID-19 comprising administration of an effective amount of an ATR inhibitor to the subject. In one aspect of this embodiment, the subject has been previously vaccinated with a SARS-CoV-2 vaccine and develops vaccine-related exacerbation of infection, for example, an antibody-dependent enhancement or related antibody-mediated mechanisms of vaccine/antibody-related exacerbation.

In any of the above embodiments, the administration of the ATR inhibitor results in one or more clinical benefits to the subject. In one aspect of this embodiment, the one or more clinical benefits is shortening the duration of infection, reduction of the likelihood of hospitalization, reduction in the likelihood of mortality, reduction in the likelihood of ICU admission, reduction in the likelihood being placed on mechanical ventilation, reduction in the likelihood supplemental oxygen will be needed, and/or reduction in the length of hospital stay. In a further aspect of this embodiment, the one or more clinical benefit is the failure of the subject to develop significant symptoms of COVID-19.

The compounds of the invention can be administered before or following an onset of SARS-CoV-2 infection, or after acute infection has been diagnosed in a subject. The aforementioned compounds and medical products of the inventive use are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g. in order to boost the response and eradicate the pathogens and/or symptoms of the disease. The methods of the invention can also be used to reduce the likelihood of developing a disorder or even prevent the initiation of disorders associated with COVID-19 in advance of the manifestation of mild to moderate disease, or to treat the arising and continuing symptoms of an acute infection.

Treatment of mild to moderate COVID-19 is typically done in an outpatient setting. Treatment of moderate to severe COVID-19 is typically done inpatient in a hospital setting. Additionally, treatment can continue in an outpatient setting after a subject has been discharged from the hospital.

The invention furthermore relates to a medicament comprising at least one compound according to the invention or a pharmaceutically salts thereof.

A “medicament” in the meaning of the invention is any agent in the field of medicine, which comprises one or more compounds of the invention or preparations thereof (e.g. a pharmaceutical composition or pharmaceutical formulation) and can be used in prophylaxis, therapy, follow-up or aftercare of patients who suffer from clinical symptoms and/or known exposure to COVID-19.

### Combination Treatment

In various embodiments, the active ingredient may be administered alone or in combination with one or more additional therapeutic agents. A synergistic or augmented effect may be achieved by using more than one compound in the pharmaceutical composition. The active ingredients can be used either simultaneously or sequentially.

In one embodiment, the ATR inhibitor is administered in combination with one or more additional therapeutic agents. In one aspect of this embodiment, the one or more additional therapeutic agents is selected from anti-inflammatories, antibiotics, anti-coagulants, antiparasitic agent, antiplatelet agents and dual antiplatelet therapy, angiotensin converting enzyme (ACE) inhibitors, angiotensin II receptor blockers, beta-blockers, statins and other combination cholesterol lowering agents, specific cytokine inhibitors, complement inhibitors, anti-VEGF treatments, JAK inhibitors, immunomodulators, anti-inflammasome therapies, sphingosine-1 phosphate receptors binders, N-methyl-d-aspartate (NDMA) receptor glutamate receptor antagonists, corticosteroids, Granulocyte-macrophage colony-stimulating factor (GM-CSF), anti-GM-CSF, interferons, angiotensin receptor-neprilysin inhibitors, calcium channel blockers, vasodilators, diuretics, muscle relaxants, and antiviral medications.

In one embodiment, the ATR inhibitor is administered in combination with an antiviral agent. In one aspect of this embodiment, the antiviral agent is remdesivir. In another aspect of this embodiment, the antiviral agent is lopinavir-ritonavir, alone or in combination with ribavirin and interferon-beta.

In one embodiment, the ATR inhibitor is administrated in combination with a broad-spectrum antibiotic.

In one embodiment, the ATR inhibitor is administered in combination with chloroquine or hydroxychloroquine. In one aspect of this embodiment, the ATR inhibitor is further combined with azithromycin.

In one embodiment, the ATR inhibitor is administered in combination with interferon-1-beta (Rebif®).

In one embodiment, the ATR inhibitor is administered in combination with one or more additional therapeutic agents selected from hydroxychloroquine, chloroquine, ivermectin, tranexamic acid, nafamostat, virazole, ribavirin, lopinavir/ritonavir, favipiravir, arbidol, leronlimab, interferon beta-1a, interferon beta-1b, beta-interferon, azithromycin, nitrazoxamide, lovastatin, clazakizumab, adalimumab, etanercept, golimumab, infliximab, sarilumab, tocilizumab, anakinra, emapalumab, pirfenidone, belimumab, rituximab, ocrelizumab, anifrolumab, ravulizumab-cwvz, eculizumab, bevacizumab, heparin, enoxaparin, apremilast, coumadin, baricitinib, ruxolitinib, dapafliflozin, methotrexate, leflunomide, azathioprine, sulfasalazine, mycophenolate mofetil, colchicine, fingolimod, ifenprodil, prednisone, cortisol, dexamethasone, methylprednisolone, melatonin, otilimab, ATR-002, APN-01, camostat mesylate, brilacidin, IFX-1, PAX-1-001, BXT-25, NP-120, intravenous immunoglobulin (IVIG), and solnatide.

In one embodiment, the ATR inhibitor is administered in combination with one or more anti-inflammatory agent. In one aspect of this embodiment, the anti-inflammatory agent is selected from corticosteroids, steroids, COX-2 inhibitors, and non-steroidal anti-inflammatory drugs (NSAID). In one aspect of this embodiment, the anti-inflammatory agent is diclofenac, etodolac, fenoprofen, flurbirprofen, ibuprofen, indomethacin, meclofenamate, mefenamic acid, meloxicam, nabumetone, naproxen, oxaprozin, piroxicam, sulindac, tolmetin, celecoxib, prednisone, hydrocortisone, fludocortisone, bethamethasone, prednisolone, triamcinolone, methylprednisone, dexamethasone, fluticasone, and budesonide (alone or in combination with formoterol, salmeterol, or vilanterol).

In one embodiment, the ATR inhibitor is administered in combination with one or more immune modulators. In one aspect of this embodiment the immune modulator is a calcineurin inhibitor, antimetabolite, or alkylating agent. In another aspect of this embodiment, the immune modulator is selected from azathioprine, mycophenolate mofetil, methotrexate, dapson, cyclosporine, cyclophosphamide, and the like.

In one embodiment, the ATR inhibitor is administered in combination with one or more antibiotics. In one aspect of this embodiment, the antibiotic is a broad-spectrum antibiotic. In another aspect of this embodiment, the antibiotic is a pencillin, anti-straphylococcal penicillin, cephalosporin, aminopenicillin (commonly administered with a betalactamase inhibitor), monobactam, quinoline, aminoglycoside, lincosamide, macrolide, tetracycline, glycopeptide, antimetabolite or nitroimidazole. In a further aspect of this embodiment, the antibiotic is selected from penicillin G, oxacillin, amoxicillin, cefazolin, cephalexin, cephotetan, cefoxitin, ceftriazone, augmentin, amoxicillin, ampicillin (plus sulbactam), piperacillin (plus tazobactam), ertapenem, ciprofloxacin, imipenem, meropenem, levofloxacin, moxifloxacin, amikacin, clindamycin, azithromycin, doxycycline, vancomycin, Bactrim, and metronidazole.

In one embodiment, the ATR inhibitor is administered in combination with one or more anti-coagulants. In one aspect of this embodiment, the anti-coagulant is selected from apixaban, dabigatran, edoxaban, heparin, rivaroxaban, and warfarin.

In one embodiment, the ATR inhibitor is administered in combination with one or more antiplatelet agents and/or dual antiplatelet therapy. In one aspect of this embodiment, the antiplatelet agent and/or dual antiplatelet therapy is selected from aspirin, clopidogrel, dipyridamole, prasugrel, and ticagrelor.

In one embodiment, the ATR inhibitor is administered in combination with one or more ACE inhibitors. In one aspect of this embodiment, the ACE inhibitor is selected from benazepril, captopril, enalapril, fosinopril, lisinopril, moexipril, perindopril, quinapril, ramipril and trandoliapril.

In one embodiment, the ATR inhibitor is administered in combination with one or more angiotensin II receptor blockers. In one aspect of this embodiment, the angiotensin II receptor blocker is selected from azilsartan, candesartan, eprosartan, irbesartan, losartan, olmesartan, telmisartan, and valsartan.

In one embodiment, the ATR inhibitor is administered in combination with one or more beta-blockers. In one aspect of this embodiment, the beta-blocker is selected from acebutolol, atenolol, betaxolol, bisoprolol/hydrochlorothiazide, bisoprolol, metoprolol, nadolol, propranolol, and sotalol.

In another embodiment, the ATR inhibitor is administered in combination with one or more alpha and beta-blocker. In one aspect of this embodiment, the alpha and beta-blocker is carvedilol or labetalol hydrochloride.

In one embodiment, the ATR inhibitor is administered in combination with one or more interferons.

In one embodiment, the ATR inhibitor is administered in combination with one or more angiotensin receptor-neprilysin inhibitors. In one aspect of this embodiment, the angiotensin receptor-neprilysin inhibitor is sacubitril/valsartan.

In one embodiment, the ATR inhibitor is administered in combination with one or more calcium channel blockers. In one aspect of this embodiment, the calcium channel blocker is selected from amlodipine, diltiazem, felodipine, nifedipine, nimodipine, nisoldipine, and verapamil.

In one embodiment, the ATR inhibitor is administered in combination with one or more vasodilators. In one aspect of this embodiment, the one or more vasodilator is selected from isosorbide dinitrate, isosorbide mononitrate, nitroglycerin, and minoxidil.

In one embodiment, the ATR inhibitor is administered in combination with one or more diuretics. In one aspect of this embodiment, the one or more diuretics is selected from acetazolamide, amiloride, bumetanide, chlorothiazide, chlorthalidone, furosemide, hydrochlorothiazide, indapamide, metalozone, spironolactone, and torsemide.

In one embodiment, the ATR inhibitor is administered in combination with one or more muscle relaxants. In one aspect of this embodiment, the muscle relaxant is an antispasmodic or antispastic. In another aspect of this embodiment, the one or more muscle relaxants is selected from casisoprodol, chlorzoxazone, cyclobenzaprine, metaxalone, methocarbamol, orphenadrine, tizanidine, baclofen, dantrolene, and diazepam.

In one embodiment, the ATR inhibitor is administered in combination with one or more antiviral medications. In one aspect of this embodiment, the antiviral medication is remdesivir.

In one embodiment, the ATR inhibitor is administered in combination with one or more additional therapeutic agents selected from antiparasitic drugs (including, but not limited to, hydroxychloroquine, chloroquine, ivermectin), antivirals (including, but not limited to, tranexamic acid, nafamostat, virazole [ribavirin], lopinavir/ritonavir, favipiravir, leronlimab, interferon beta-1a, interferon beta-1b, beta-interferon), antibiotics with intracellular activities (including, but not limited to azithromycin, nitrazoxamide), statins and other combination cholesterol lowering and anti-inflammatory drugs (including, but not limited to, lovastatin), specific cytokine inhibitors (including, but not limited to, clazakizumab, adalimumab, etanercept, golimumab, infliximab, sarilumab, tocilizumab, anakinra, emapalumab, pirfenidone), complement inhibitors (including, but not limited to, ravulizumab-cwvz, eculizumab), anti-VEGF treatments (including, but not limited to, bevacizumab), anti-coagulants (including, but not limited to, heparin, enoxaparin, apremilast, coumadin), JAK inhibitors (including, but not limited to, baricitinib, ruxolitinib, dapafliflozin,), anti-inflammasone therapies (including, but not limited to, colchicine), sphingosine-1 phosphate receptors binders (including, but not limited to, fingolimod), N-methyl-d-aspartate (NDMA) receptor glutamate receptor antagonists (including, but not limited to, ifenprodil), corticosteroids (including, but not limited to, prednisone, cortisol, dexamethasone, methylprednisolone), GM-CSF, anti-GM-CSF (otilimab), ATR-002, APN-01, camostat mesylate, arbidol, brilacidin, IFX-1, PAX-1-001, BXT-25, NP-120, intravenous immunoglobulin (IVIG), and solnatide.

In some embodiments, the combination of a ATR inhibitor with one or more additional therapeutic agents reduces the effective amount (including, but not limited to, dosage volume, dosage concentration, and/or total drug dose administered) of the ATR inhibitor and/or the one or more additional therapeutic agents administered to achieve the same result as compared to the effective amount administered when the ATR inhibitor or the additional therapeutic agent is administered alone. In some embodiments, the combination of a ATR inhibitor with the additional therapeutic agent reduces the total duration of treatment compared to administration of the additional therapeutic agent alone. In some embodiments, the combination of an ATR inhibitor with the additional therapeutic agent reduces the side effects associated with administration of the additional therapeutic agent alone. In some embodiments, the combination of an effective amount of the ATR inhibitor with the additional therapeutic agent is more efficacious compared to an effective amount of the ATR inhibitor or the additional therapeutic agent alone. In one embodiment, the combination of an effective amount of the ATR inhibitor with the one or more additional therapeutic agent results in one or more additional clinical benefits than administration of either agent alone.

As used herein, the terms “treatment,” “treat,” and “treating” refer to reversing, alleviating, delaying the onset of, or inhibiting the progress of a viral infection, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a known exposure to an infected person and/or in light of comorbidities which are predictors for severe disease, or other susceptibility factors).

### EXEMPLIFICATION

### EXAMPLE 1: ANTIVIRAL TESTING

### Viral Replication Kinetics

Human lung adenocarcinoma epithelial cells (Calu-3) were seeded in 24-well plates with 3.5 x 105 cells/ml, 1 ml per well, for 24 h. The compounds to be tested were diluted in Coronavirus (MERS, SARS-CoV-1 or SARS-CoV-2) infection medium to reach the final concentrations. The growth medium was removed from the cells, cells were washed once with 1× PBS (phosphate buffered saline), and subsequently inoculated with Coronavirus at a MOI (multiplicity of infection) of 0.01. After attachment of viral particles to the cells for 45 min, the inoculum was removed, cells were washed twice with 1× PBS, and infection medium containing compounds was added (1 ml/well). As Coronavirus replication peaks at approximately 48 h post infection, this time point was chosen for all subsequent analyses. At 48 h p.i., supernatants were collected from infected cells and stored at −80° C. Then, viral titers were determined by plaque test on African green monkey kidney epithelial cells (VeroE6) cells as described below.

### Cell Viability Assay

Calu-3 were seeded in 96-well plates with 3.5×105 cells/ml, 100 μl per well, for 24 h. The compounds to be tested or pure DMSO as positive control were serially diluted in SARS-CoV- 2 infection medium (DMEM, supplemented with 1% L-Glu, 1% P/S and 2% FBS) to obtain 5- fold of the desired final concentrations. The growth medium was removed from the cells and replaced with 80 μl/well of fresh infection medium. Subsequently, 20 μl of the diluted compounds were added in quadruplicates for each concentration (i.e. 5-fold dilution to reach the final concentrations). Cells were incubated for 48 h at 37° C. (5% CO2, 96% rH). At 48 h post treatment, cell viability was measured on a Tecan Safire 2 plate reader using the CellTiter 96® Non-Radioactive Cell Proliferation Assay (MTT) (Promega) according to manufacturer's instructions.

### Plaque Test

Viral titers in supernatants collected from infected cells were determined by plaque test on VeroE6 cells. Briefly, VeroE6 cells were seeded in 12-well plates (1:6 dilution of a confluent flask), 1.5 ml/well, for 24 h. Cell culture supernatants were 10-fold serially diluted in 1× PBS. The growth medium was removed from the cells, cells were washed once with 1× PBS, and diluted supernatants were added (150 μl/well). After 30 min inoculation, an overlay medium (double-concentrated minimal essential medium (MEM; supplemented with 2% L-Glu, 2% P/S, 0.4% bovine serum albumin (BSA)), mixed 1:1 with 2.5% avicel solution (prepared in ddH2O) was added to the cells (1.5 ml/well). Then, cells were incubated for 72 h at 37° C. After 72 h, the overlay medium was removed from the cells, and following a washing step with 1x PBS the cells were fixed with 4% paraformaldehyde (PFA) for at least 30 min at 4° C. Subsequently, the 4% PFA solution was removed, and the cells were counterstained with crystal violet solution to visualize the virus-induced plaques in the cell layer. The number of plaques at a given dilution was used to calculate the viral titers as plaque-forming units (PFU/ml).

### Statistics

All statistical evaluations were performed using GraphPad Prism 8 (v4.8.3). Statistical significant differences in viral titers were determined using a non-parametric t-test (Mann-Whitney Test). IC50 and maximum effect values were obtained by fitting a sigmoidal curve onto the data of an eight-point dose response curve experiment.

### Compound Testing and Results

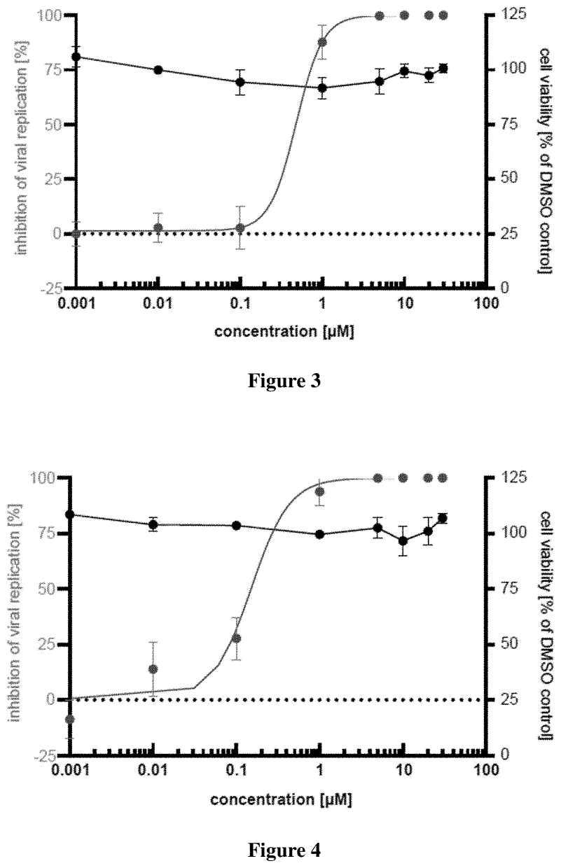

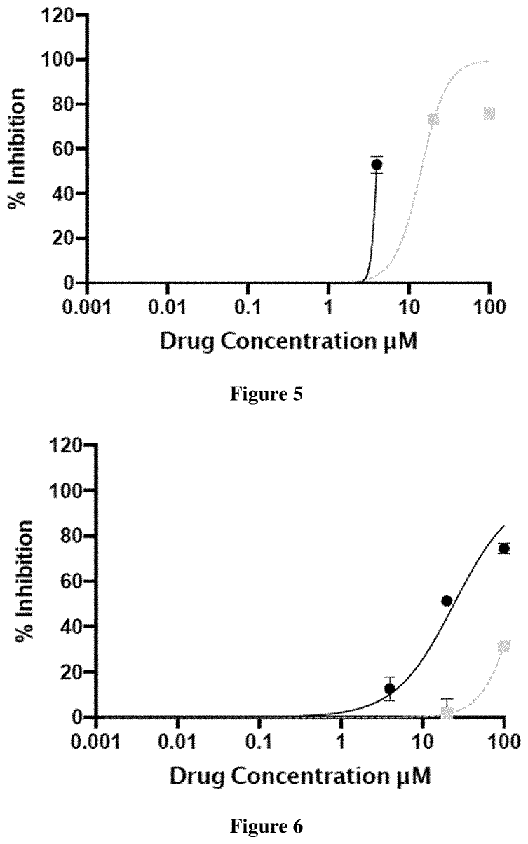

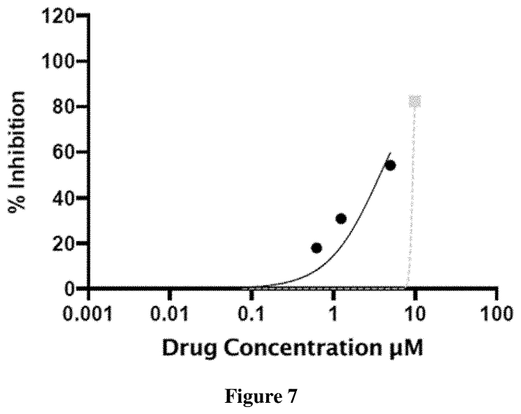

Using the methods described above influence of Compound 1 on viral replication of Coronavirus MERS, SARS-CoV-1 and SARS-CoV-2 and on cell viability was tested. In addition, influence on viral replication of SARS-CoV-2 and on cell viability of remdesivir was tested as a reference (antiviral compound remdesivir is one of the most promising candidates for the treatment of COVID-19). The results obtained with Compound 1 on the viral replication in Calu-3 cells infected with MERS are shown in FIG. 1, the results obtained with Compound 1 on the viral replication in Calu-3 cells infected with SARS-CoV-1 are shown in FIG. 2, results obtained with Compound 1 on the viral replication in Calu-3 cells infected with SARS-CoV-2 are shown in FIG. 3 and results obtained with remdesivir on the viral replication in Calu-3 cells infected with SARS-CoV-2 are shown in FIG. 4. As apparent from FIGS. 1 to 3 Compound 1 leads to a dose dependent inhibition of virus replication of all Coronaviruses tested (MERS, SARS-CoV-1 and SARS-CoV-2) whereby in each case the cell viability remains nearly unaffected. Surprisingly, the inhibition induced by Compound 1 in cells infected by SARS-CoV-2 is comparable with that of the antiviral reference substance remdesivir.

### EXAMPLE 2: ANTIVIRAL TESTING—CYTOMEGALOVIRUS

To determine the antiviral activity of the compounds, human foreskin fibroblasts (HFF) were treated with a 5-fold serial dilution of each compound ranging from 100 μM to 0.0128 μM for 1h before infection. Antiviral activity was determined five days later, using an immunofluorescence-based assay. Cytotoxicity was determined using an MTT assay on uninfected cells treated with the same concentrations of compound and for the same length of time. Acyclovir was included as an assay control.

### Experimental Procedure

The antiviral activity of 8 dilutions of each compounds was explored following administration 1h before infection with HCMV. Compound and virus were left on the cells for the entire duration of the experiment (5 days). The cytotoxicity of the same range of concentrations of compounds was determined by MTT assay.

### Cell Plating

Cells were seeded in complete media (DMEM (Gibco, 61965026) supplemented with 10% FBS (Gibco 10500064) and 1× p/s (Gibco 15070063) at 4,000 cells/100 μl well in four 96 well plates: two for the cytotoxicity assay and two for the infectivity assay. After seeding, the plates were incubated at RT for 5 minutes for even distribution, and then at 37° C., 5% CO2 until the following day. Compound 1 and Control (Acyclovir) were diluted from 10 mM stock solutions 1:50 to 200 μM in supplemented media (DMEM (Gibco, 61965026) supplemented with 5% FBS (Gibco 10500064) and 1× p/s (Gibco 15070063), and 2250 of these diluted stocks or diluent only (1% DMSO) were added in triplicate to the top raw (A) of a round bottom 96 well plate.

180 μl of 0.2% DMSO diluent were added in all other wells (rows B-H). In this way, the percentage of DMSO was kept constant at 0.2% across the serial dilution. Only in row A the concentration of DMSO was 1% (also in the uninfected/ untreated controls), reflecting the DMSO concentration in the first dilution from the stock. A five-fold serial dilution was performed by transferring 45 μl from row A into row B, mixing, and then again from row B into C etc. until row H.

### Pre-treatment of Cells

50 μl of supplemented media per well were added to the cells in each plate (infectivity and cytotoxicity). 50 μl per well of treatment from the dilution plate were transferred to the cells in corresponding positions in each plate (infectivity and cytotoxicity). All plates were incubated at 37° C., 5% CO2.

### Infection

The virus stock (HCMV Merlin strain, 1×10⁶ IU/ml) was diluted 5-fold with supplemented media to bring the concentration to 2×10⁵ IU/ml. After 1 h pre-treatment, media/treatment was removed from the cells and 50 μl per well of treatment from the dilution plate were re-transferred to the cells in corresponding positions in the infectivity plates. 50 μl virus per well (MOI ˜1) were added, except the uninfected control, where 50 μl of supplemented media without virus were added.

### Fixation and Development

After five days, the infected plates were washed with PBS, fixed for 30 mins with 4% formaldehyde, washed again with PBS, and stored in PBS at 4° C. overnight until staining. The cytotoxicity plates were treated with MTT to determine cell viability.

### Infectivity Readout

Cells were immunostained. For that any residual formaldehyde was quenched with 50 mM ammonium chloride, after which cells were permeabilised (0.1% Triton X100) and stained with an antibody recognizing HCMV gB (The Native Antigen Company). The primary antibody was detected with an Alexa-488 conjugate secondary antibody (Life Technologies, A21207), and nuclei were stained with Hoechst. Images were acquired on an Opera Phenix high content confocal microscope (Perkin Elmer) using a 10× objective, and percentage infection calculated using Columbus software (infected cells/total cells×100).

### Cytotoxicity Readout

Cytotoxicity was detected by MTT assay. For that the MTT reagent (Sigma, M5655) was added to the cells for 2 h at 37° C., 5% CO2, after which the media was removed and the precipitate solubilised with a mixture of 1:1 Isopropanol:DMSO for 20 minutes. The supernatant was transferred to a clean plate and signal read at 570 nm.

1. A method of treating a coronavirus infection in a subject in need thereof, comprising administering an effective amount of an ATR inhibitor, or a pharmaceutically acceptable salt thereof, to the subject.

2. The method of claim 1, wherein the coronavirus causes a SARS or MERS infection.

3. The method of claim 1, wherein the coronavirus causes a SARS-CoV-1 or SARS-CoV-2 or MERS-CoV infection.

4. The method of claim 1, wherein the coronavirus is SARS-CoV-2.

5. The method of claim 1, wherein the ATR inhibitor is 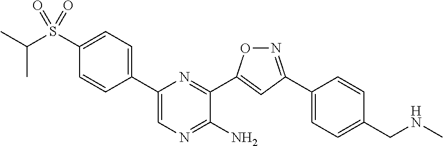 [SMILES: CNCc1ccc(-c2cc(-c3nc(-c4ccc(S(=O)(=O)C(C)C)cc4)cnc3N)on2)cc1]
or a pharmaceutically acceptable salt thereof.

6. The method of claim 1, wherein the administration of the ATR inhibitor results in the reduction of the viral load in the subject.

7. The method of claim 1, wherein the ATR inhibitor reduces or inhibits the virus induced activation of the DNA damage response in the infected cells.

8. The method of claim 1, wherein the ATR inhibitor is administered prior to COVID-19 pneumonia development.

9. The method of claim 1, wherein the subject has a mild to moderate SARS-CoV-2 infection.

10. The method of claim 1, wherein the subject has been previously vaccinated with a SARS-CoV-2 vaccine and develops vaccine-related exacerbation of infection, for example, an antibody-dependent enhancement or related antibody-mediated mechanisms of vaccine/antibody-related exacerbation.

11. The method of claim 1, wherein the subject is asymptomatic at the start of the treatment.

12. The method of claim 1, wherein the subject has had known contact with a patient who has been diagnosed with a SARS-CoV-2 infection.

13. The method of claim 1, wherein the subject begins administration of the ATR inhibitor prior to being formally diagnosed with SARS-CoV-2 infection.

14. The method of claim 1, wherein the administration of the ATR inhibitor results in one or more clinical benefits.

15. The method of claim 14, wherein the one or more clinical benefits is selected from: shortening the duration of infection, reduction of the likelihood of hospitalization, reduction in the likelihood of mortality, reduction in the likelihood of ICU admission, reduction in the likelihood being placed on mechanical ventilation, reduction in the likelihood supplemental oxygen will be needed, and/or reduction in the length of hospital stay.

16. The method of claim 1, wherein the subject is undergoing outpatient treatment.

17. The method of claim 1, further comprising administration of one or more additional therapeutic agent.

18. The method of claim 17, wherein the one or more additional therapeutic agents is selected from anti-inflammatories, antibiotics, anti-coagulants, antiparasitic agent, antiplatelet agents and dual antiplatelet therapy, angiotensin converting enzyme (ACE) inhibitors, angiotensin II receptor blockers, beta-blockers, statins and other combination cholesterol lowering agents, specific cytokine inhibitors, complement inhibitors, anti-VEGF treatments, JAK inhibitors, immunomodulators, anti-inflammasome therapies, sphingosine-1 phosphate receptors binders, N-methyl-d-aspartate (NDMA) receptor glutamate receptor antagonists, corticosteroids, Granulocyte-macrophage colony-stimulating factor (GM-CSF), anti-GM-CSF, interferons, angiotensin receptor-neprilysin inhibitors, calcium channel blockers, vasodilators, diuretics, muscle relaxants, and antiviral medications.

19. The method of claim 17, wherein the one or more additional therapeutic agents is an antiviral medication.

20. The method of claim 17, wherein the one or more additional therapeutic agents is remdesivir.

21. The method of claim 17, wherein the one or more additional therapeutic agents is lopinavir-ritonavir.

22. The method of claim 17, wherein the one or more additional therapeutic agents further includes ribavirin and interferon-beta.

23. The method of claim 17, wherein the one or more additional therapeutic agents is chloroquine or hydroxychloroquine.

24. The method of claim 17, wherein the one or more additional therapeutic agents further includes azithromycin.

25. The method of claim 17, wherein the one or more additional therapeutic agents is interferon-1-beta (Rebif®).

26. The method of claim 17, wherein the one or more additional therapeutic agent is selected from hydroxychloroquine, chloroquine, ivermectin, tranexamic acid, nafamostat, virazole [ribavirin], lopinavir/ritonavir, favipiravir, leronlimab, interferon beta-1a, interferon beta-1b, beta-interferon, azithromycin, nitrazoxamide, lovastatin, clazakizumab, adalimumab, etanercept, golimumab, infliximab, sarilumab, tocilizumab, anakinra, emapalumab, pirfenidone, ravulizumab-cwvz, eculizumab, bevacizumab, heparin, enoxaparin, apremilast, coumadin, baricitinib, ruxolitinib, dapafliflozin, colchicine, fingolimod, ifenprodil, prednisone, cortisol, dexamethasone, methylprednisolone, GM-CSF, otilimab, ATR-002, APN-01, camostat mesylate, arbidol, brilacidin, IFX-1, PAX-1-001, BXT-25, NP-120, intravenous immunoglobulin (WIG), and solnatide.

27. The method of claim 1, wherein the ATR inhibitor is administered between about 20 mg to about 2000 mg, which is applied once to four times per day to once every week.

28. The method of claim 1, wherein the total amount of ATR inhibitor administered is between about 50 mg and about 350 mg per day.

29. The method of claim 1, wherein the ATR inhibitor is administered for about 7 days to about 21 days.

30. The method of claim 1, wherein the ATR inhibitor is administered orally.
